(19) United States
(12) Patent Application Publication
(10) Pub. No.: US 2020/0188004 A1
(43) Pub. Date: Jun. 18, 2020

(54) FASTENER REMOVAL TOOLS

(71) Applicant: Spinal Elements, Inc., Carlsbad, CA (US)

(72) Inventor: Markanthony Flores, Chula Vista, CA (US)

(21) Appl. No.: 16/717,859

(22) Filed: Dec. 17, 2019

Related U.S. Application Data
(60) Provisional application No. 62/780,886, filed on Dec. 17, 2018.

(51) Int. Cl. A61B 17/88

(52) U.S. Cl. CPC A61B 17/8872; A61B 17/7082; A61B 17/8888

(57) ABSTRACT

A fastener removal tool has a proximal end, a distal end, and a rotatable and translatable body between the proximal end and the distal end. There is a spring positioned between the proximal end and the distal end, wherein the spring is configured to allow the rotatable and translatable body to translate. Rotation and translation of the rotatable and translatable body is configured to provide a compressive force to at least a portion of a fastener for removal of the fastener. Methods for using fastener removal tools are also disclosed.

### CROSS-REFERENCE TO RELATED APPLICATIONS

This application claims priority benefit to U.S. Provisional Patent Application No. 62/780,886, filed Dec. 17, 2018, which is hereby incorporated by reference herein in its entirety.

### FIELD

The present application relates generally to fastener removal tools. More particularly, the present application relates to tools for removing fasteners from bone.

### BACKGROUND

The spinal column generally comprises thirty-three vertebrae. The vertebrae help provide anatomical support and stabilization. Dysfunction or damage to the vertebrae may require surgery to alleviate any corresponding disability, compression, instability and/or pain. Surgery may include the use of fixation techniques, which may include the use of rods, plates and/or screws to affix vertebral support devices to bone using fasteners.

Variations in bone density and/or surgical technique may result in poor affixation of the fasteners into bone. Bone surrounding a fastener may be of poor quality and insufficient to hold the fastener in place. Bone may also be stripped and lose the ability to retain a fastener. A different fastener or alternative method may be employed to address such issues. Removal of a fastener may also be required during surgery or post-surgery to replace or remove an implanted device. For example, a bone screw may be removed from stripped bone due to poor affixation to employ a different sized screw or alternative implanted device for use in a procedure.

Tools exist for removing fasteners from bone and include tools that employ torqueing, clamping, and grabbing to remove fasteners. However, a need still exists for a tool that removes fasteners from bone in an elegant and efficient manner.

### SUMMARY

In some embodiments, a fastener removal tool is provided. The fastener removal tool can include an external shaft comprising a proximal end and a distal end. The fastener removal tool can include a driver tip extending distally from the distal end of the external shaft, wherein the driver tip comprises driver arms. The fastener removal tool can include an internal shaft residing within the external shaft. The fastener removal tool can include a retention shaft extending distally from the distal end of the internal shaft, wherein the retention shaft comprises retention features. The fastener removal tool can include a shaft spring disposed about the retention shaft between the internal shaft and the driver tip, wherein at least a portion of the retention features and at least a portion of the driver arms are insertable into at least a portion of a fastener with the retention features and driver tips in an aligned configuration, and further wherein the shaft spring provides a force to translate the retention shaft.

In some embodiments, the external shaft and internal shaft are rotatable relative to each other. In some embodiments, the retention shaft is translatable with the retention features and driver tips in a non-aligned configuration. In some embodiments, the force of the shaft spring is configured to direct or translate the retention features and the driver arms towards each other. In some embodiments, the distal end of the retention shaft is positioned distally to the distal end of the driver tip in an unlocked position for ready engagement with a fastener. In some embodiments, the distal end of the retention shaft is positioned proximally to the distal end of the driver tip in a locked position for secure engagement with a fastener. In some embodiments, the retention tips and the driver arms are aligned in an unlocked position for ready engagement with a fastener. In some embodiments, the retention tips and the driver arms are non-aligned in a locked position for secure engagement with a fastener. In some embodiments, the retention features are slideably engageable underneath a surface element of a fastener without release of the shaft spring. In some embodiments, the retention features comprise retention tips extending laterally from the distal end of the retention shaft.

In some embodiments, a method of using a fastener removal tool is provided. The method can include delivering at least a portion of a distal end of the fastener removal tool within at least a portion of a fastener. The method can include rotating a rotatable and translatable shaft of the fastener removal tool, wherein rotating positions the distal end of the fastener removal tool underneath a surface element of the fastener. The method can include translating the rotatable and translatable shaft to secure the fastener to the fastener removal tool, wherein a spring facilitates the translating and provides a compression force to secure the fastener. The method can include withdrawing the fastener removal tool and the fastener.

In some embodiments, the distal end of the fastener removal tool comprises driver arms and the rotatable and translatable shaft comprises retention features. In some embodiments, the retention features comprise retention tips extending laterally from the distal end of the rotatable and translatable shaft. In some embodiments, rotating moves the driver arms and retention features from an aligned position to a non-aligned position. In some embodiments, the driver arms and retention features are aligned in an unlocked position for ready engagement with a fastener. In some embodiments, the driver arms and retention features are non-aligned in a locked position for secure engagement with a fastener. In some embodiments, translating transitions the distal end of the driver arms from a position proximal to the retention features to a position distal to the retention features. In some embodiments, the retention features are positioned underneath the surface element of the fastener in the non-aligned position. In some embodiments, the method can include positioning the distal end of the rotatable and translatable shaft distally to a distal end of a driver tip of the fastener removal tool in an unlocked position for ready engagement with a fastener. In some embodiments, the method can include positioning the distal end of the rotatable and translatable shaft proximally to a distal end of a driver tip of the fastener removal tool in a locked position for secure engagement with a fastener.

### BRIEF DESCRIPTION OF THE DRAWINGS

These and other features, aspects and advantages of the described embodiments are described with reference to drawings of certain preferred embodiments, which are intended to illustrate, but not to limit. It is to be understood that the attached drawings are for the purpose of illustrating concepts of the described embodiments and may not be to scale.

FIG. 1 is a side view of an embodiment of a fastener removal tool in an unlocked position.

FIG. 2 is a rotated side view of the fastener removal tool of FIG. 1.

FIG. 3 is a rotated side view of the shaft assembly of FIG. 1.

FIG. 3A is a rotated side view of the shaft assembly of FIG. 3.

FIG. 3B is a side view of the shaft assembly of FIG. 3A in a locked position.

FIG. 4 is a top view of the shaft assembly of FIG. 3.

FIG. 5 is a side view of an embodiment of components of a shaft assembly.

FIG. 6 is a side view of an embodiment of components of a shaft assembly.

FIG. 7 is a side view of an embodiment of components of a shaft assembly.

FIG. 8 is an exploded top view of an embodiment of a hub assembly.

FIG. 9 is a side view of an embodiment of a retention shaft.

FIG. 10 is a close-up view of element 10-10 of the retention shaft of FIG. 9.

FIG. 11 is a cross-section view of the retention shaft of FIG. 9 along the line 11-11 of FIG. 9.

FIG. 12 is a close-up view of element 12-12 of the retention shaft of FIG. 11.

FIG. 13 is a side view of an embodiment of a driver tip.

FIG. 14 is a bottom view of the driver tip of FIG. 13.

FIG. 15 is a cross-section view of the driver tip of FIG. 14 along the line 15-15 of FIG. 14.

FIG. 16 is a cross-section view of the driver tip of FIG. 15 along the line 16-16 of FIG. 15.

FIG. 17 is a top view of an embodiment of an external shaft.

FIG. 18 is a side view of the external shaft of FIG. 17.

FIG. 19 is a rotated side view of the external shaft of FIG. 18.

FIG. 20 is a cross-section view of the proximal end of the external shaft of FIG. 17 along the line 20-20 of FIG. 17.

FIG. 21 is a cross-section view of the proximal end of the external shaft of FIG. 17 along the line 21-21 of FIG. 17.

FIG. 22 is a view of element 22-22 of FIG. 17.

FIG. 23 is a close-up view of element 23-23 of FIG. 18.

FIG. 24 is a close-up view of element 24-24 of FIG. 19.

FIG. 25 is a proximal end view of an embodiment of an internal shaft.

FIG. 26 is a cross-section view of the internal shaft of FIG. 25 along the line 26-26 of FIG. 25.

FIG. 27 is a close-up view of element 27-27 of FIG. 26.

FIG. 28 is a side view of an embodiment of a hub assembly.

FIG. 29 is a cross-section view of the hub assembly of FIG. 28 along the line 29-29 of FIG. 28.

FIG. 30 is a cross-section view of the hub assembly of FIG. 28 along the line 30-30 of FIG. 28.

FIG. 31 is a top view of an embodiment of a weld ring.

FIG. 32 is a cross-section view of the weld ring of FIG. 31 along the line 32-32 of FIG. 31.

FIG. 33 is a side view of an embodiment of a quick connect component.

FIG. 34 is a rotated side view of the quick connect component of FIG. 33.

FIG. 35 is a top view of an embodiment of a button.

FIG. 36 is a side view of the button of FIG. 35.

FIG. 37 is a cross-section view of the button of FIG. 36 along the line 37-37 of FIG. 36.

### DETAILED DESCRIPTION

As will be explained herein, certain embodiments of the disclosed fastener removal tools provide advantages over current tools. For example, the fastener removal tools disclosed herein may be configured for rotation and/or translation with features that allow secure engagement of at least a portion of a fastener using the rotation and/or translation features and allow for removal of the fastener in an elegant and efficient manner.

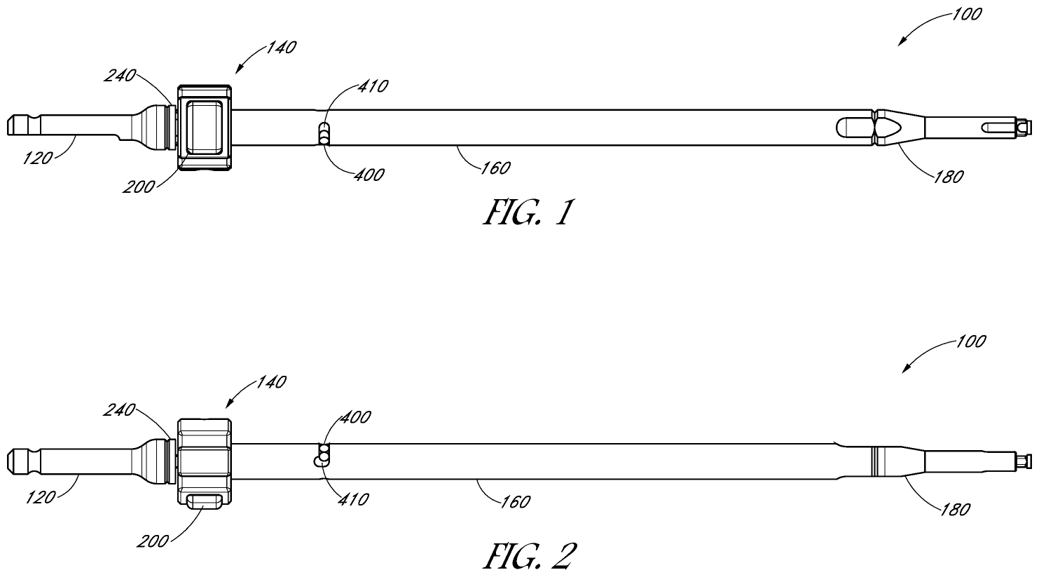

FIG. 1 illustrates an embodiment of a fastener removal tool 100. Fastener removal tool 100 may be used to remove fasteners such as screws, bolts, or other fasteners, from bone, tissue, or other materials. For example, fastener removal tool 100 may be used to remove a screw during or after surgery from bone that has poor bone quality, such as increased porosity, or from bone that has become stripped, and lost its ability to securely retain a screw. In such instances, a different type or size of screw may be employed, or the permanent removal of the screw may be desired. Fastener removal tool 100 may be used as an alternative or additional tool to facilitate removal of a screw, which provides advantages over use of the same device used to insert the screw. For example, a device used to insert a screw may employ a mechanism for driving a screw into bone, which is not designed for removal of the screw in the best manner. Fastener removal tool 100 may be any length to facilitate removal of a fastener. Various dimensions, proportions and/or angles may be incorporated into the design of fastener removal tool 100 and are contemplated within the scope of the present disclosure.

Fastener removal tool 100 can include a quick connect component 120, weld ring 240, hub assembly 140, external shaft 160, and driver tip 180. Quick connect component 120 is positioned at a proximal end of fastener removal tool 100 and driver tip 180 is positioned at a distal end of fastener removal tool 100. Quick connect component 120 may be engaged with weld ring 240 and/or external shaft 160. A handle may be attached to quick connect component 120.

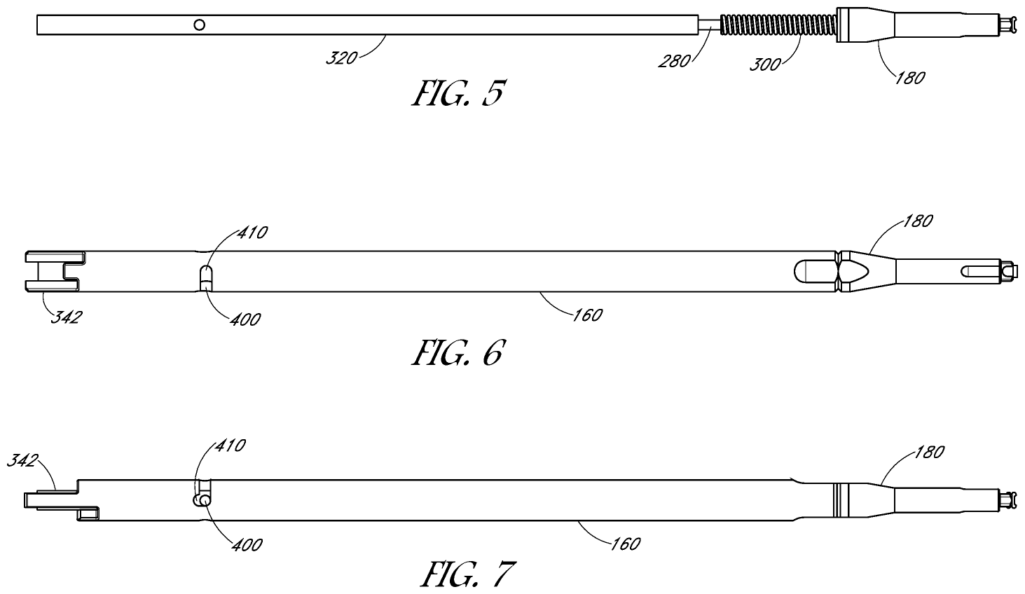

In the embodiment of FIG. 1, fastener removal tool 100 is shown in an unlocked position. FIG. 2 illustrates a rotated view of fastener removal tool 100 in an unlocked position, wherein driver tip 180 is shown in a different position. Button 200 on hub assembly 140 can be pressed to allow external shaft 160 and/or internal shaft 320 (as shown in FIG. 5) to rotate with respect to each other, wherein rotation is confined by the travel of pin 400 within pin slot 410 of external shaft 160. Pin 400 may be positioned on one or both sides of internal shaft 320 or through internal shaft 320, in order to provide added stability and strength to pin 400. Rotation allows fastener removal tool 100 to rotate from an unlocked starting position to a locked ending position. In an embodiment, the distal end of internal shaft 320 can be positioned distally to the distal end of the driver tip 180 in an unlocked position for ready engagement with a fastener. In an embodiment, the distal end of internal shaft 320 can be positioned via rotation and translation proximally to the distal end of the driver tip 180 in a locked position for secure engagement with a fastener.

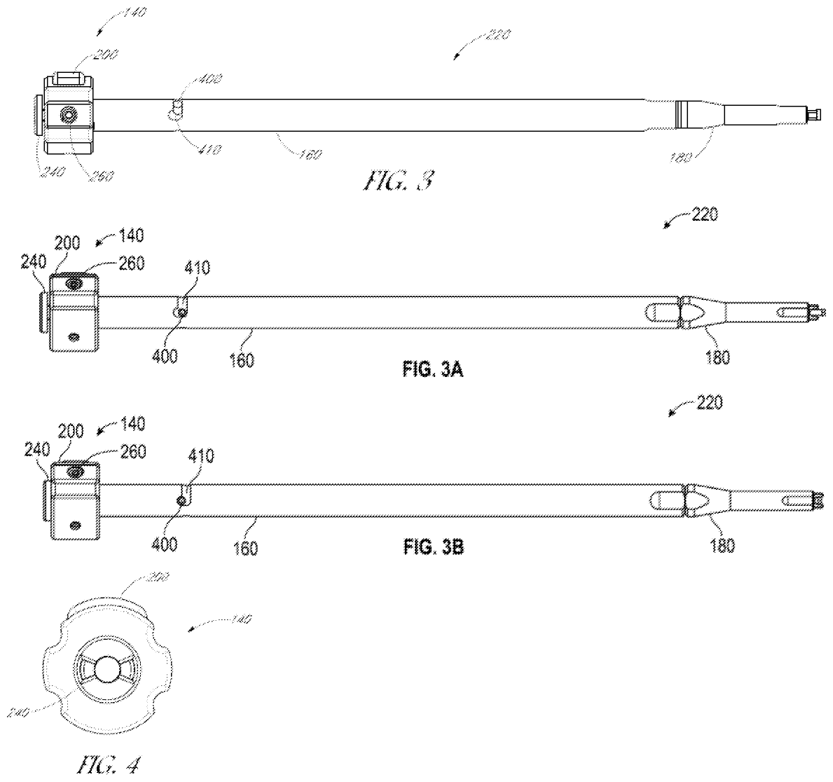

FIG. 3 illustrates shaft assembly 220 of fastener removal tool 100. Shaft assembly 220 includes weld ring 240, hub assembly 140, external shaft 160 and driver tip 180. Fastener element 260 secures button 200 to hub assembly 140. Hub assembly 140 is attached to external shaft 160. FIG. 3A illustrates a rotated side view of shaft assembly 220 showing the distal end of retention shaft 280 (as shown in FIG. 5) in a non-aligned position relative to the distal end of driver tip 180, prior to translation of retention shaft 280 relative to external shaft 160. FIG. 3B illustrates a side view of shaft assembly 220 showing the distal end of retention shaft 280 in a non-aligned position relative to the distal end of driver tip 180, subsequent to translation of retention shaft 280 relative to external shaft 160.

FIG. 4 illustrates a top view of shaft assembly 140 showing hub assembly 140, button 200 and weld ring 240. The hub assembly 140 is shown with an outer shape configured for tactile engagement by a user.

FIG. 5 illustrates an embodiment of components of shaft assembly 220, including retention shaft 280, shaft spring 300, driver tip 180, and internal shaft 320. Shaft spring 300 is disposed about retention shaft 280 between driver tip 180 and internal shaft 320. Internal shaft 320 engages with shaft spring 300 to provide biasing of shaft spring 300 between the proximal end of driver tip 180 and the distal end of internal shaft 320. As internal shaft 320 abuts shaft spring 300, the proximal end of retention shaft 280 may be fixedly attached to the distal end of internal shaft 320, whereby shaft spring 300 provides a consistent and/or constant force.

In the illustrated example, the spring-loaded feature provided by shaft spring 300 allows a consistent and/or constant force to be applied to engage and retain a fastener via the distal end of driver tip 180 and the distal end of retention shaft 280. In the unlocked starting position, the distal end of retention shaft 280 is distal to the distal end of driver tip 180. Upon rotation of internal shaft 320 and/or external shaft 160, the distal end of retention shaft 280 can be rotated to allow proximal translation of retention shaft 280 and internal shaft 320, wherein the distal end of retention shaft 280 translates to a position proximal to the distal end of driver tip 180. As translated, pin 400 travels proximally within pin slot 410 facilitated by the force exerted by shaft spring 300. Components such as locking and/or unlocking features for maintaining and/or releasing a fastener are also contemplated within the scope of this disclosure.

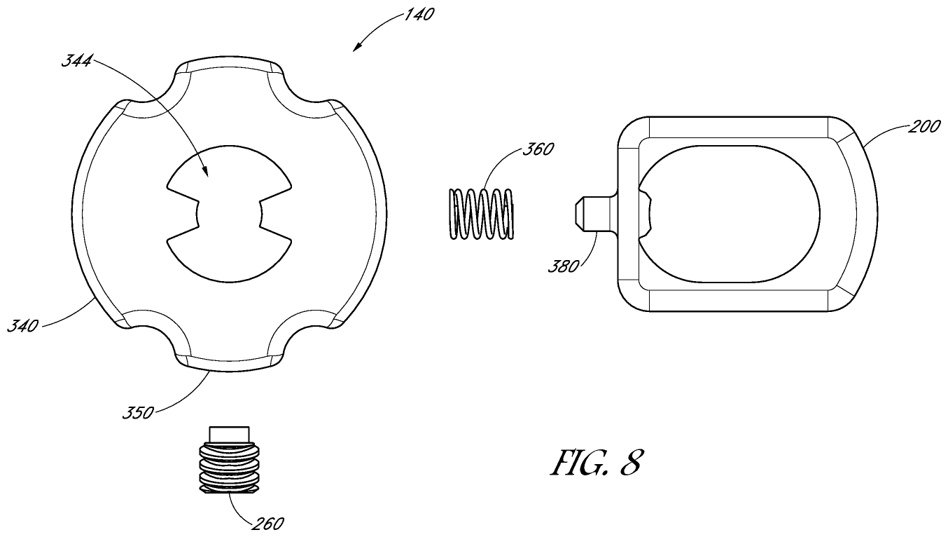

FIGS. 6-7 further illustrate components of shaft assembly 220, including external shaft 160 with the proximal end of external shaft 160 configured to engage and couple with hub assembly 140. The proximal end of external shaft 160 comprises hub adaptor 342, which engages with internal opening 344 of hub 340 (as shown in FIG. 8) in order to couple hub assembly 140 to the proximal end of external shaft 160, whereby hub assembly 140 is configured to rotate about hub adaptor 342 and external shaft 160. Hub adaptor 342 may be configured to allow a rotation angle of 90° within internal opening 344 of hub 340, although other configurations are also contemplated within the scope of this disclosure. For example, hub adaptor 342 may be configured to allow for a rotation angle of 45° or any other angle to accommodate the design of hub adaptor 342 and internal opening 344 of hub 340. Fastener removal tool 100 may be configured to allow hub adaptor 342 to rotate within internal opening 344 of hub 340 or may be configured to allow internal opening 344 of hub 340 to rotate around hub adaptor 342. In an embodiment, as hub adaptor 342 rotates within internal opening 344 of hub 340, external shaft 160 may correspondingly rotate, thereby facilitating rotation of the distal end of driver tip 180 to facilitate engagement with a fastener. In an embodiment, as internal opening 344 of hub 340 rotates around hub adaptor 342, internal shaft 320 and retention shaft 280 may correspondingly rotate, thereby facilitating rotation of the distal end of retention shaft 280 to facilitate engagement with a fastener.

Referencing FIGS. 5-7, the spring-loaded feature provided by shaft spring 300 is created by inserting the proximal end of retention shaft 280 therethrough into the distal end of driver tip 180, disposing shaft spring 300 about retention shaft 280, towards the distal end of retention shaft 280 and towards driver tip 180. Shaft spring 300 may be configured to abut against at least a portion of driver tip 180, for example, disposed within driver tip 180. Internal shaft 320 is then disposed about retention shaft 280, such that the proximal end of retention shaft 280 engages and/or contacts the distal end of internal shaft 320. Shaft spring 300 is then compressed between internal shaft 320 and driver tip 180. Retention shaft 280 may be fixedly attached, for example via weld, to internal shaft 320. External shaft 160 is disposed about internal shaft 320, shaft spring 300, and retention shaft 280. External shaft 160 may be fixedly attached, for example via weld, to driver tip 180. Pin 400 may be press-fit into one or more openings in internal shaft 320, with pin 400 tangent to the outside surface of external shaft 160.

FIG. 8 illustrates an exploded top view of an embodiment of hub assembly 140 featuring hub 340, hub spring 360, button 200, and fastener element 260. Hub spring 360 is disposed about button connector 380. Button 200 and hub spring 360 engage with hub 340 via button connector 380 through an opening (not shown) in hub 340. Button connector 380 and hub spring 360 may be partially or entirely disposed within hub 340. Button 200 engages within hub 340 via fastener element 260 via threaded opening 350 (not shown) as the hub spring 360 is compressed. Fastener element 260 may be a set screw or other fastener. Fastener element 260 may be disposed against the surface of and/or within button 200 partially or entirely within hub 340. Other configurations and components for engaging button 200 with hub assembly 140 are also contemplated within the scope of this disclosure.

Hub assembly 140 may be rotatably coupled to external shaft 160, with weld ring 240 proximal to hub assembly 140, wherein weld ring 240 is fixedly attached to external shaft 160 to confine hub assembly 140 between weld ring 240 and external shaft 160, wherein hub assembly 140 is allowed to rotate and translate about external shaft 160. Hub assembly 140 may be configured to rotate 90° upon deployment and/or operation of button 200, although other configurations are also contemplated within the scope of this disclosure. In the illustrated embodiment, button 200 restricts rotation unless desired by deployment and/or operation of button 200. Upon deployment and/or operation of button 200, internal shaft 320 and retention shaft 280 are configured to allow for translation and/or rotation relative to external shaft 160 along a path defined by the travel of pin 400 within pin slot 410.

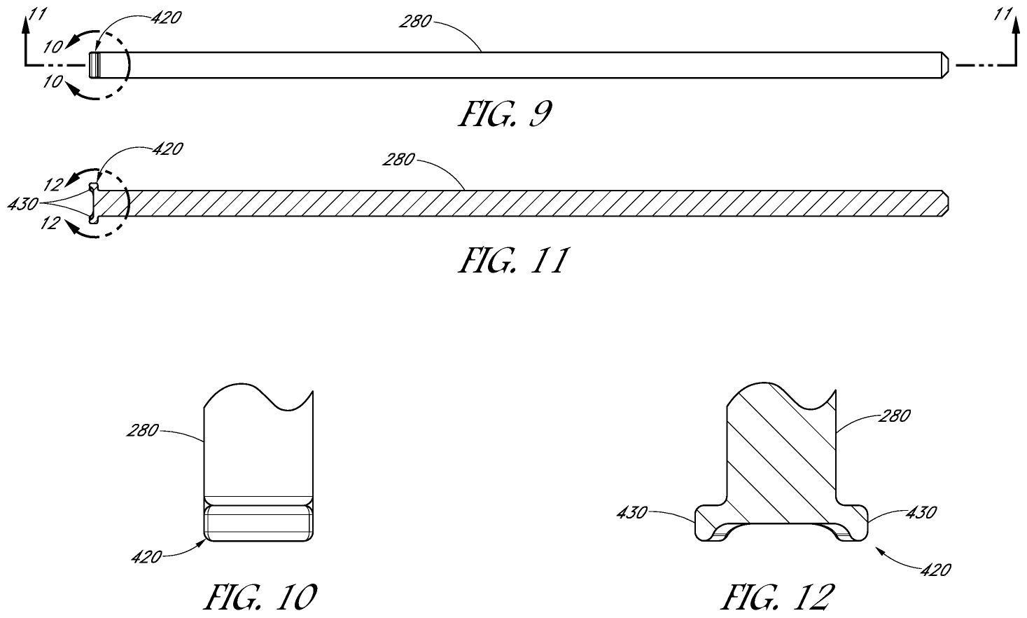

FIGS. 9-12 illustrate the retention shaft 280 and distal end of retention shaft 280 in further detail. Distal end of retention shaft 280 comprises retention feature 420. Retention feature 420 further comprises retention tips 430. Retention tips 430 may be suitably shaped to deploy and engage a corresponding screw, screw surface, screw tulip and/or other feature. At least a portion of retention feature 420 may engage with a screw and/or screw surface with at least a portion of one or more retention tips 430. At least a portion of one or more retention tips 430 may engage with a screw surface underneath the screw surface. Any suitable number and/or shape of retention tips 430 is contemplated within the scope of this disclosure. For example, 2, 3, 4, 5, or 6 retention tips 430 may be suitable for a corresponding fastener design. For further example, rectangular, triangular, or polygonal retention tips 430 may be suitable for a corresponding fastener design. In an embodiment, retention feature 420 may be fully disposed within a fastener, for example a screw head and/or screw tulip, to facilitate retention of the screw. In an embodiment, the distal end of driver tip 180 may be fully disposed within a fastener, for example a screw head and/or screw tulip, to facilitate retention of the screw. Retention feature 420 may be configured to rotate, for example, from a resting angle of 0° to a rotated angle of 90°, relative to the distal end of driver tip 180, within the screw head and/or screw tulip.

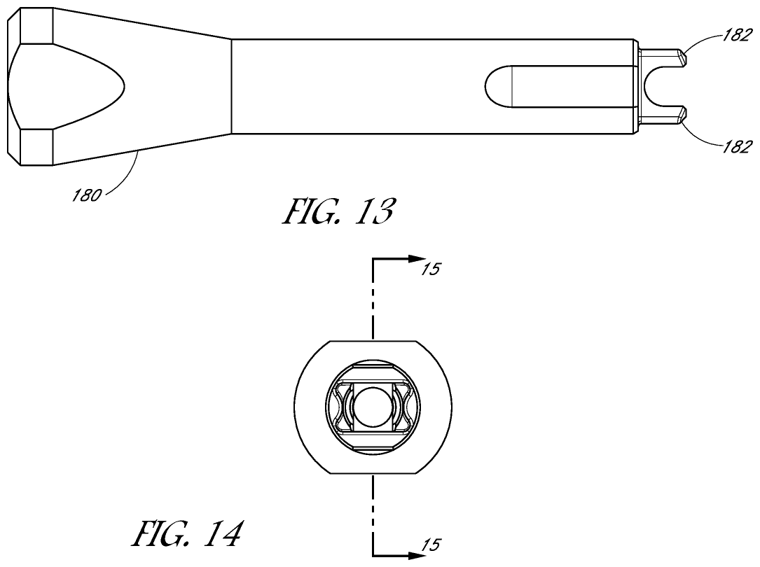

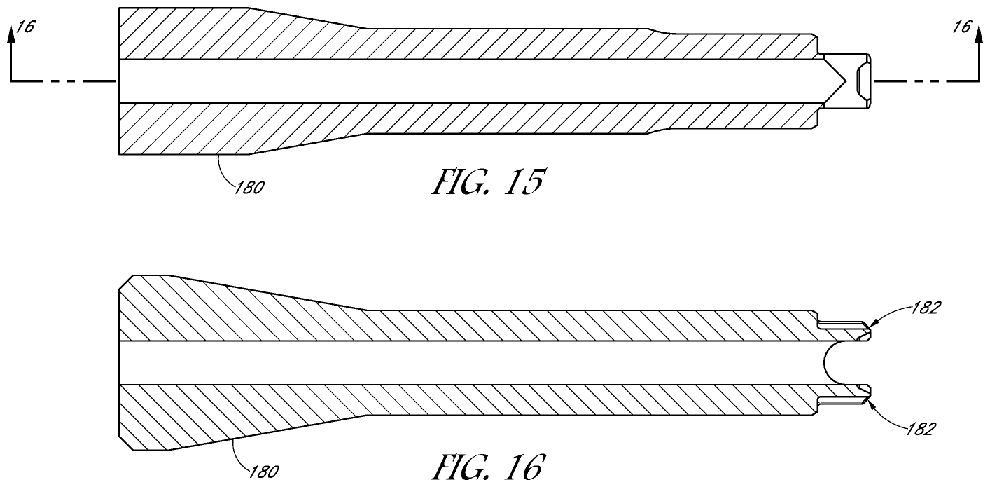

FIGS. 13-16 illustrate driver tip 180. Distal end of driver tip 180 further comprises driver arms 182. As the aforementioned retention feature 420 of the distal end of retention shaft 280, as discussed in connection with FIG. 8 and FIGS. 9-12, is translated beyond the distal end of driver tip 180, retention shaft 280 may be rotated 90° to transition retention tips 430 from a non-aligned position to an aligned position, relative to driver arms 182. Retention tips 430 and driver arms 182 may be held apart by compression of shaft spring 300. Release of shaft spring 300 provides a compressive force directing or translating retention tips 430 and driver arms 182 towards each other, thereby facilitating engagement of retention tips 430 and driver arms 182 with at least a portion of a fastener. Retention tips 430 and driver arms 182 may alternatively be slideably engaged with an element of a fastener, such that retention tips 430 and driver arms 182 are positioned into contact with at least a portion of a fastener without release of shaft spring 300. In an embodiment, retention tips 430 and driver arms 182 may alternatively be slideably engaged with an element of a fastener to position retention tips 430 underneath a surface element of a fastener, such that retention tips 430 are positioned into contact with the surface element of the fastener without release of shaft spring 300. In an embodiment, the retention features are slideably engageable underneath a surface element of a fastener without release of shaft spring 300.

In various embodiments, the fastener removal tool 100 of the present disclosure may be used for removal of fasteners, such as those disclosed in U.S. Pat. No. 8,100,955 entitled “Orthopedic Expansion Fastener” assigned to Spinal Elements, Inc. and issued Jan. 24, 2012, which is hereby incorporated for reference in its entirety for any and all purposes.

As an example of the aforementioned steps, distal end of retention shaft 280 and distal end of driver tip 180 may be at least partially disposed or fully disposed within at least a portion of a fastener. In an embodiment, retention shaft 280 comprises retention tips 430, wherein the retention tips 430 extend laterally from the distal end of the retention shaft 280. Retention shaft 280 may then be rotated 90°, relative to the distal end of driver tip 180, within the at least a portion of the fastener to be positioned underneath a surface element of the fastener, for example a clip within a fastener, such that retention tips 430 and driver arms 182 at the distal end of driver tip 180 are transitioned from an aligned position to a non-aligned position, wherein the retention tips 430 are positioned underneath the surface element of the fastener. Upon release of shaft spring 300 via translation of retention shaft 280, retention tips 430 and driver arms 182 may be directed or translated towards one another. As a result, retention tips 430 provide a compression force against the surface element of the fastener, thereby allowing fastener removal tool 100 to engage, retain, and withdraw the fastener. Thereafter, the fastener may be disengaged from fastener removal tool 100 by deployment and/or operation of button 200 and released by translation and/or rotation of retention shaft 280 with internal shaft 320 via movement of pin 400 within pin slot 410.

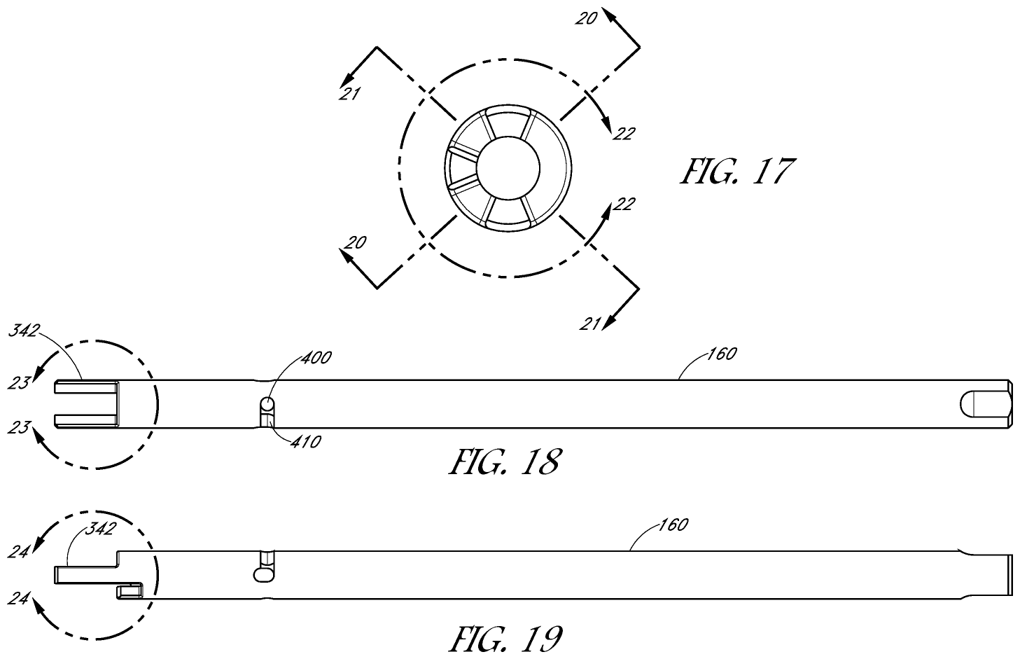

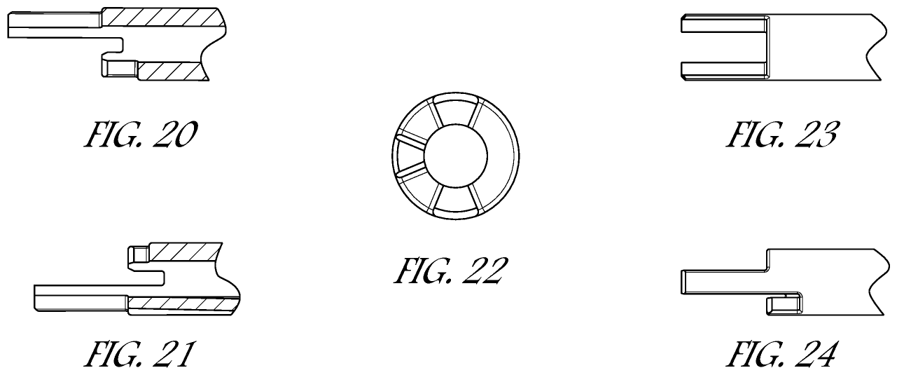

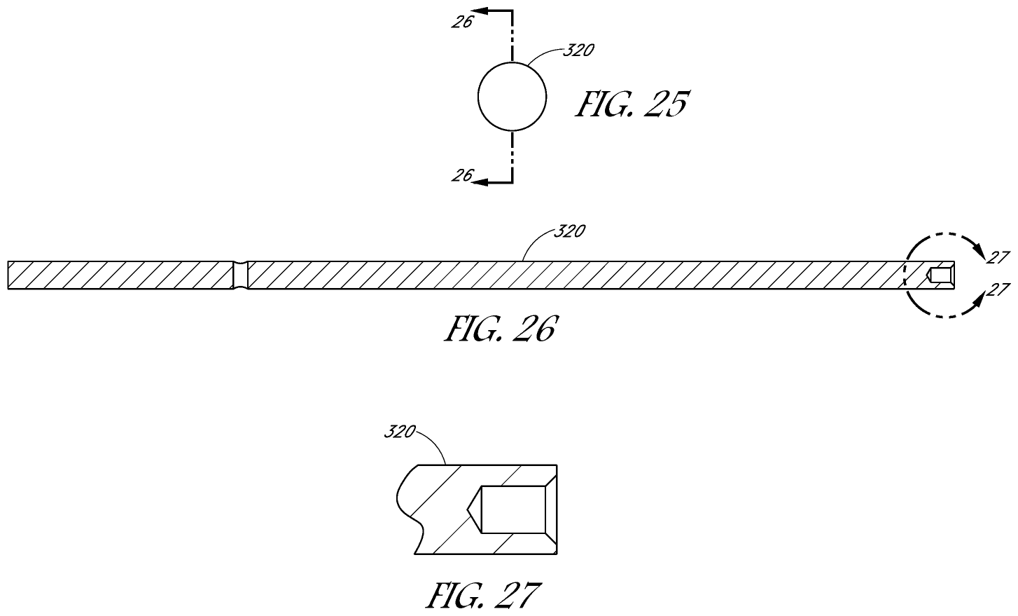

FIGS. 17-24 illustrate an embodiment of external shaft 160. The proximal end of external shaft 160 is configured to engage with hub assembly 140, weld ring 240 and/or quick connect component 120. Various configurations of engagement of the proximal end of external shaft 160 are contemplated within the scope of this disclosure. Engagement of hub assembly 140, weld ring 240 and/or quick connect component 120 with external shaft 160 may be configured to allow for rotation of the proximal end of external shaft 160 or the proximal end of internal shaft 320 to correspondingly rotate the distal end of external shaft 160 or the distal end of internal shaft 320.

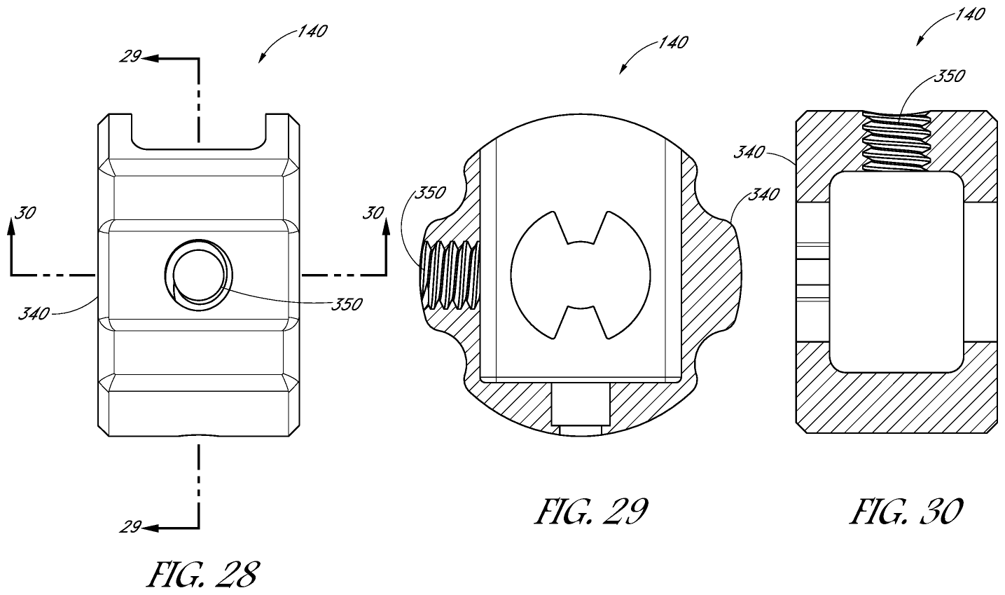

FIGS. 25-27 illustrate an embodiment of internal shaft 320 and distal end of internal shaft 320 in further detail. In an embodiment, the distal end of internal shaft 320 is configured to engage with the proximal end of retention shaft 280 and may be fixedly attached thereto. Distal end of internal shaft 320 may be configured in a “U” or “V” shaped distal end.

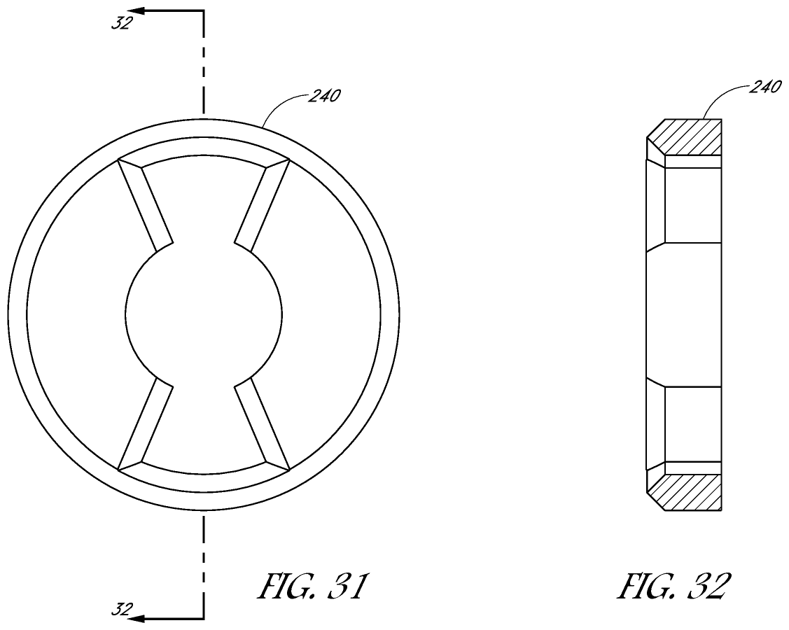

FIGS. 28-30 illustrate hub assembly 140 in further detail. FIG. 28 shows hub 340 with threaded opening 350. FIG. 29 shows a cross-section view of hub 340 depicting an internal portion of the threaded opening 350 configured for accepting insertion of fastener element 260 (not shown) against and/or within button 200 (not shown). FIG. 30 shows a further cross-section view of hub 340 with threaded opening 350.

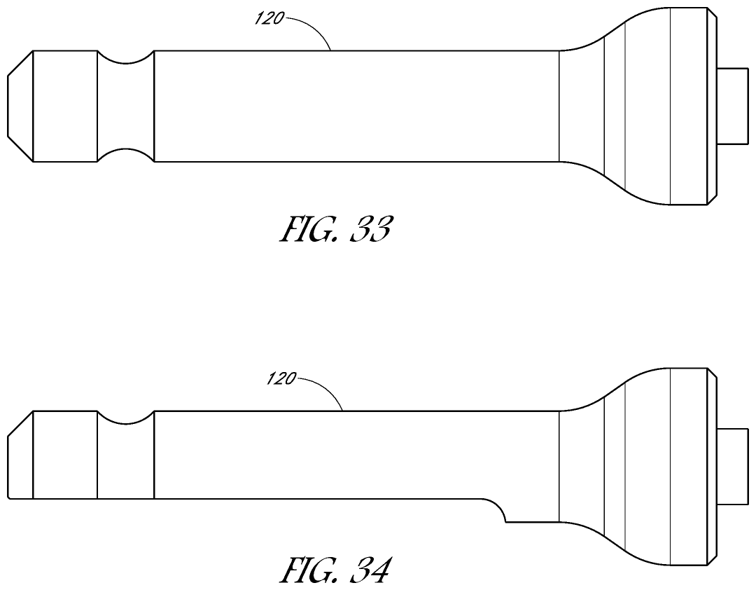

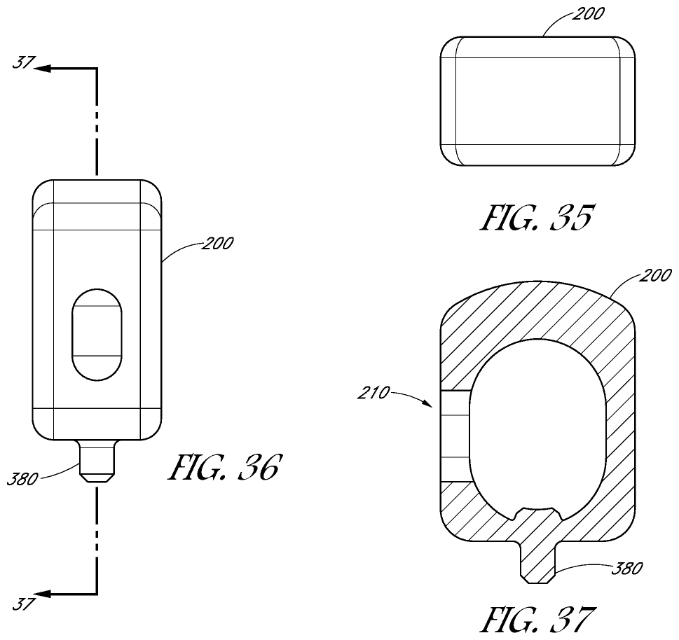

FIGS. 31-32 illustrate top and cross-section views of the proximal end of weld ring 240. FIGS. 33-34 illustrate a quick connect component 120 for attaching a handle to shaft assembly 220. FIGS. 35-37 illustrate an embodiment of button 200, depicting button connector 380, for use with hub assembly 140. Fastener element 260 may inserted against and/or within button 200 via button slot 210.

While certain embodiments have been shown and described herein, it will be apparent to those skilled in the art that such embodiments are provided by way of example only. Numerous variations, changes, and substitutions are within the scope of this disclosure and will be apparent to those skilled in the art as within the scope of the invention. It should be understood that alternatives to the embodiments described herein may be employed. It is furthermore intended that the following claims define the scope of the invention and that structures and methods within the scope of these claims and their equivalents be covered thereby.

1. A fastener removal tool comprising:
an external shaft comprising a proximal end and a distal end;
a driver tip extending distally from the distal end of the external shaft, wherein the driver tip comprises driver arms;
an internal shaft residing within the external shaft;
a retention shaft extending distally from the distal end of the internal shaft, wherein the retention shaft comprises retention features;
a shaft spring disposed about the retention shaft between the internal shaft and the driver tip;
wherein at least a portion of the retention features and at least a portion of the driver arms are insertable into at least a portion of a fastener with the retention features and driver tips in an aligned configuration; and
further wherein the shaft spring provides a force to translate the retention shaft.

2. The fastener removal tool of claim 1, wherein the external shaft and internal shaft are rotatable relative to each other.

3. The fastener removal tool of claim 1, wherein the retention shaft is translatable with the retention features and driver tips in a non-aligned configuration.

4. The fastener removal tool of claim 1, wherein the force of the shaft spring is configured to direct or translate the retention features and the driver arms towards each other.

5. The fastener removal tool of claim 1, wherein the distal end of the retention shaft is positioned distally to the distal end of the driver tip in an unlocked position for ready engagement with a fastener.

6. The fastener removal tool of claim 1, wherein the distal end of the retention shaft is positioned proximally to the distal end of the driver tip in a locked position for secure engagement with a fastener.

7. The fastener removal tool of claim 1, wherein the retention tips and the driver arms are aligned in an unlocked position for ready engagement with a fastener.

8. The fastener removal tool of claim 1, wherein the retention tips and the driver arms are non-aligned in a locked position for secure engagement with a fastener.

9. The fastener removal tool of claim 1, wherein the retention features are slideably engageable underneath a surface element of a fastener without release of the shaft spring.

10. The fastener removal tool of claim 1, wherein the retention features comprise retention tips extending laterally from the distal end of the retention shaft.

11. A method of using a fastener removal tool, comprising:
delivering at least a portion of a distal end of the fastener removal tool within at least a portion of a fastener;
rotating a rotatable and translatable shaft of the fastener removal tool, wherein rotating positions the distal end of the fastener removal tool underneath a surface element of the fastener;
translating the rotatable and translatable shaft to secure the fastener to the fastener removal tool, wherein a spring facilitates the translating and provides a compression force to secure the fastener; and
withdrawing the fastener removal tool and the fastener.

12. The method of using a fastener removal tool of claim 11, wherein the distal end of the fastener removal tool comprises driver arms and the rotatable and translatable shaft comprises retention features.

13. The method of using a fastener removal tool of claim 11, wherein the retention features comprise retention tips extending laterally from the distal end of the rotatable and translatable shaft.

14. The method of using a fastener removal tool of claim 11, wherein rotating moves the driver arms and retention features from an aligned position to a non-aligned position.

15. The method of using a fastener removal tool of claim 11, wherein the driver arms and retention features are aligned in an unlocked position for ready engagement with a fastener.

16. The method of using a fastener removal tool of claim 11, wherein the driver arms and retention features are non-aligned in a locked position for secure engagement with a fastener.

17. The method of using a fastener removal tool of claim 11, wherein translating transitions the distal end of the driver arms from a position proximal to the retention features to a position distal to the retention features.

18. The method of using a fastener removal tool of claim 11, wherein the retention features are positioned underneath the surface element of the fastener in the non-aligned position.

19. The method of using a fastener removal tool of claim 11, further comprising positioning the distal end of the rotatable and translatable shaft distally to a distal end of a driver tip of the fastener removal tool in an unlocked position for ready engagement with a fastener.

20. The method of using a fastener removal tool of claim 11, further comprising positioning the distal end of the rotatable and translatable shaft proximally to a distal end of a driver tip of the fastener removal tool in a locked position for secure engagement with a fastener.
